(19) United States
(12) Patent Application Publication
(10) Pub. No.: US 2014/0350349 A1
(43) Pub. Date: Nov. 27, 2014

(54) HISTORY LOG OF USERS ACTIVITIES AND ASSOCIATED EMOTIONAL STATES

(71) Applicant: KONINKLIJKE PHILIPS N.V., EINDHOVEN (NL)

(72) Inventors: Lucas Jacobus Franciscus Geurts, Best (NL); Remco Timmer, Utrecht (NL); Tjadde Rutgher Brouwer, Laren (NL); Theodorus Gerardus Hugo Cornelius Raaijmakers, Amsterdam (NL); Wendelina Elise Corinne Loopik, Utrecth (NL)

(73) Assignee: KONINKLIJKE PHILIPS. N.V., EINDHOVEN (NL)

(21) Appl. No.: 14/365,333

(22) PCT Filed: Dec. 5, 2012
(86) PCT No.: PCT/IB2012/056990

§ 371 Date: Jun. 13, 2014

Related U.S. Application Data
(60) Provisional application No. 61/576,569, filed on Dec. 16, 2011.

(51) Int. Cl. A61B 5/00; A61B 5/11; A61B 5/16

(52) U.S. Cl. CPC A61B 5/0022; A61B 5/165; A61B 5/1112; A61B 5/1118; A61B 5/486; A61B 5/7475
USPC 600/300

(57) ABSTRACT

Data about a person's activity and about the person's emotional state during the activity is logged in a history log. The history log may serve as a diary to this person. The history log of one or more persons may serve as a reference-profile for generating recommendations.

### FIELD OF THE INVENTION

The invention relates to a method of providing a service on a data network. The invention also relates to a network-enabled device, and to a personal electronic device.

### BACKGROUND ART

The Internet has enabled people to find individuals complying with a certain pre-determined profile much more quickly than before the advent of the Internet. For example, virtual communities have come to the foreground wherein people share their knowledge, feelings or opinions via the Internet. The expression “virtual community” refers to a social network of individuals who interact via specific media such as chat rooms, social networking websites, message boards, microblogs, websites posting customer-satisfaction ratings of enterprises, virtual worlds, massive multi-player gaming, etc. The expression “social network” refers to a group of individuals that forms a social structure based on interdependences, or commonalities, between the individuals. Examples of such interdependencies or commonalities are friendship, kinship, shared interests, shared beliefs, attending the same event, etc. The term “blog” is a blend of the expression “web log” and refers to a type of website, which is maintained by an individual and at which the individual posts updates from time to time in the form of commentary, descriptions of events, or other material such as graphics or video. The term “microblog” refers to a blog featuring very short postings.

An example of a community service provided on the Internet is described in US patent application publication 20050004923, entitled “Community service providing system and method”, filed for Hyuncheol Park, and incorporated herein by reference. The abstract of US patent application publication 20050004923 refers to a system and method for providing community services wherein emotions or conditions of respective communication service users can be indirectly confirmed by representing information on the emotions or conditions of predetermined communication service users as coordinate values on an emotion map configured on the basis of predetermined information axes and generating predetermined events in accordance with determination results of similarities or differences between the coordinate values based on the coordinate values represented on the emotion map, and thus, opportunities to meet other community members can also be provided through the generation of suitable events. For clarity, the feature “emotion map” as used in the description of US patent application publication 20050004923, refers to an abstract space spanned by multiple axes. Each respective one of the multiple axes refers to a particular emotion or mood. If a user selects a particular position on the map, this particular position is characterized by a set of coordinate values, each respective coordinate value being representative of an intensity of a respective mood.

For completeness, reference is made to the fields of remote physiological monitoring and of affective computing.

The field of remote physiological monitoring relates to the remote sensing of a person's vital signs. Remote physiological monitoring is used in, e.g., the field of biotelemetry, also referred to as medical telemetry. Biotelemetry involves the application of telemetry in the medical field to remotely monitor various vital signs of ambulatory patients. Remote physiological monitoring is also used in, e.g., sports and training programs. Remote physiological monitoring is further used in hazardous environments to check if someone has fallen over (e.g., firefighters, police officers, military personnel) so as to start a search-and-rescue operation if and when needed.

In the field of affective computing, it is known to use physiological monitoring in order to detect a person's emotional state by means of monitoring and analyzing the person's physiological parameters. The expression “affective computing” refers to the field of systems and devices that can recognize, interpret, process, and simulate human feeling or emotion. Affective computing is an interdisciplinary field spanning computer science, psychology, physiology and cognitive science.

### SUMMARY OF THE INVENTION

The inventors propose, among other things, a service for being provided to a virtual community based on logging information about activities of individuals and further information about emotions of the individuals and associated with the activities. The information about the emotions of a specific individual is registered over time in a history log. The further information about the activities of the specific individual is also registered in the history log. The information about the emotions of the specific individual and the further information about the activities of the specific individual are registered in the history log in a temporal relationship with one another. The history log may then serve as a diary of this specific individual, representing a timeline of activities and of emotions associated with the activities. The specific individual has access to the diary, e.g., for reading and/or editing, and may give access, e.g., read-access, to the diary, or to parts thereof, to one or more selected other persons. The diary of the specific individual may also serve as an input to a recommender system for generating recommendations with respect to future activities. The recommender system may provide the recommendations to the specific individual him/herself and/or to other individuals in the virtual community, whose emotional profiles are comparable to the emotional profile of the specific individual. The recommendations may contain an advising in favor of a particular future activity or an advising against another particular future activity.

Examples of specific activities are: attending a specific event such as a concert in a music hall, a play or show at the theater, a movie at the cinema, a sports event, a flee market, a protest march; reading a specific book or watching a movie or documentary stored on a specific DVD being played out at home, gardening, walking the dog, hiking, bicycling, traveling, having a meal, visiting a friend or one's octogenarian godmother (also one's friend), taking a nap, just sitting on the couch at home contemplating life, the universe and everything, etc.

More specifically, the invention relates to a method of providing a service on a data network. In the method, a user is being monitored by a biosensor configured for generating biosensor data associated with a particular time interval and representative of a physiological state of the user in the particular time interval. The method comprises: receiving the biosensor data via the data network; associating with the biosensor data an emotional state assumed by the user in the particular time interval; recording in a history log emotion data that is representative of the emotional state and that is associated with the particular time interval; determining activity data representative of an activity of the user taking place in the particular time interval; and recording in the history log activity data that is representative of the activity and that is associated with the particular time interval.

Accordingly, a history log is created for this specific user that represents an evolution of this user's emotions in a temporal relationship with this user's activities. The history log thus may be used as a diary, wherein information about the user's activities is logged and wherein information about the user's emotional state per particular time interval is used as annotation. For completeness, it is remarked here that per particular time interval the user may be involved in one or more activities. For example, a first one of the activities in the particular time interval comprises attending a (boring) meeting at the office, and a second activity in the same particular time interval comprises sending emails from one's laptop during the meeting, reading a good novel during the meeting, or even teaching oneself to play the banjo during the meeting.

Analysis of the user's activities within the context of the user's emotional states may reveal a pattern specific to this user in terms of correlations between activities and emotional states. For example, the history log shows that when the user is involved in a specific activity, e.g., reading a novel, or cooking a meal, or walking the dog, he/she is typically in a relaxed mood regardless of the user's activities preceding the specific activity. Assuming that the biosensor data is being received in real-time, i.e., substantially upon being generated by the biosensor, the service may recommend to this user such a specific activity when the user is determined to be in a negative mood or negative emotional state for a time period longer than a pre-determined length. As another example, history logs are maintained for a plurality of users, and the history logs maintained for users of compatible user-profiles are clustered. Assume that a person subscribes to the service and has a particular subscriber-profile that is compatible with the user-profiles of a particular cluster. Then, the service may generate recommendations to the subscribing person based on analyzing the activities in the particular cluster's history logs that are associated with positive emotional states. For instance, the particular cluster reveals that those among the monitored users of the particular cluster, who attended a specific art exhibition or who read a specific novel, were in a positive emotional state during and some time after, attending the specific art exhibition or while reading the specific novel. The service may then generate a recommendation to attend the specific art exhibition or a similar art exhibition, or to read the specific novel or a similar novel, and submit the recommendation to the subscriber at a time wherein the subscriber apparently needs some cheering up.

The biosensor data is representative of a physiological state the user in the particular time interval.

The biosensor data may be uploaded to a server of the service provider from the biosensor substantially at runtime or in real-time, i.e., as soon as the biosensor data is generated, e.g. at the end of each particular time interval. In a first example scenario, the biosensor is equipped with, or is accommodated at, or is connected to, a network-enabled data communication device that is carried along by the user in operational use of the biosensor. For example, the network-enabled data communication device comprises a mobile telephone, a smartphone or a network-enabled personal digital assistant (PDA). In a second example scenario, the environment of the user, e.g., the user's home environment, the user's car, the user's workplace, etc., is equipped with a biosensor system configured for remotely capturing the current magnitude of one or more physiological parameters. The biosensor system is configured for identifying the individual user and comprises a network-interface for uploading the biosensor data via the data network in real-time.

Alternatively, the biosensor data generated for a sequence of multiple time intervals may be accumulated at the biosensor itself or at a memory device, e.g., a solid-state memory, connected to the biosensor, or at a network-enabled data communication device accommodated at, or accommodating or connected to, the biosensor. The accumulated biosensor data may then be uploaded at a later moment in one or more batches.

Similarly, the emotional state of the user and/or the activity of the user, both associated with the particular time interval, may be determined during, or substantially upon the end of, the particular time interval. Alternatively, the emotional state and the activity pertaining to the particular time interval may be determined later, i.e., retroactively. What is relevant is that a history log is created that stores information about the user's emotional states and further information about the user's activities in a temporal relationship with the user's emotional states, for example, in a chronological order, so as to serve as an account of the user's emotionally annotated life. The account may then be used as the user's personal diary or as input to a recommender system as touched upon above and as will be discussed below in further detail.

In an embodiment of the method of the invention, the associating of the emotional state with the biosensor data comprises: receiving first context data submitted by the user via the data network and associated with the particular time interval, the first context data being representative of the emotional state as interpreted by the user; and including the first context data into the emotion data.

The emotional state of the user during the particular time interval is represented by the first context data received from the user via the data network.

The first context data may have been generated during, or close to the end of the particular time interval or is generated afterwards. Likewise, the first context data may be submitted by the user substantially during the particular time interval, e.g., during the particular time interval, and/or a few minutes after termination of the particular time-interval. Alternatively, the first context data associated with a particular time interval may be received much later.

For example, the first context data applicable to the particular time interval is received in a batch of different first context data representative of a sequence of different time intervals. The different first context data is accumulated and stored at a data recording device of the user until the opportunity arises for the user to upload the accumulated first context data to the server of the service provider. As another example, the user submits the first context data retroactively as a retrospective annotation of his/her emotional state during the particular time interval in the past. This retroactive annotating is enabled by means of e.g., the service provider logging the biosensor data in the history log and giving the user access to the history log so as to let the user add the first context data as annotations if and when convenient to the user.

The first context data may have been compiled by the user and may take the form of, e.g., a personal text message, a picture or video clip taken by the user's camera or selected from a web page on the Internet, etc. The first context data is uploaded during, or close to the end of, the particular time interval or much later by way of a retrospective emotional annotation.

Instead of being compiled by the user, the first context data as submitted by the user is pre-formatted and comprises, e.g., a particular one of a plurality of pre-determined options selectable in a menu accessible through a user-interface, e.g., a graphical user-interface or a speech-input user-interface. The user-interface may form part of the network-enabled data communication device that is carried along by the user during operational use of the biosensor. The user-interface may also form part of other network-enabled data processing equipment of the user, e.g., the user's home PC or laptop PC, or the user's smartphone. That is, the user-interface may form part of equipment, which is independent of the network-enabled data communication device that cooperates with the biosensor and uploads the biosensor data to the service provider. For example, the user-interface gives access to a menu of a plurality of pre-configured selectable options, and each specific one of the options is representative of a particular emotional state, such as for example, “angry”, “happy”, “excited”, “sad”, “anxious”, etc. The representations of the options to the user may be uniform to all users, or may be personalized per specific user. A specific emotional state associated with a specific one of the options will, however, be uniform to all users, so as to have a frame of reference regarding emotional states that is common to all users.

Accordingly, the first context data is generated by, or under control of, the user and forms an interpretation by the user of the emotional state of the user. Alternatively, or in combination with the first context data, the emotional state of the user may be inferred from the biosensor data itself. Within this context it is remarked that determining the emotional state of a person is difficult if only the values of the physiological parameter(s) are considered that is/are indicative of the physiological state of this person. Typically, the values of the physiological parameters indicate an intensity of an emotion, but not necessarily the valence of the emotion. The expression “valence of an emotion” is used in the field of psychology to characterize types of emotions. Examples of an emotion with a positive valence are happiness, contentment, kindness, etc. Examples of an emotion with a negative valence are anger, fear, irritation, sadness. See, e.g., “Emotion recognition system using short-term monitoring of physiological signals”, K. H. Kim et al., Med. Biol. Eng. Comput., 2004, 42, pp 419-427; or “A User-Modeling Approach to Build User's Psycho-Physiological Maps of Emotions using Bio-Sensors”, Olivier Villon et al., Proc. IEEE RO-MAN 2006, The 15th IEEE International Symposium on Robot and Human Interactive Communication, Hatfield, U.K., Sep. 8, 2006, pp 269-276, IEEE. The first context data as introduced above may therefore facilitate the identifying of the emotional state.

In a further embodiment of the method of the invention, the determining of the activity data, which is representative of the activity of the user in the particular time interval, comprises at least one of: i) receiving via the data network geographical data representative of a geographical location of the user during the particular time interval and determining the activity under control of the geographical location and the particular time interval; ii) receiving via the data network calendar data that is representative of an entry into an electronic calendar of the user and that is relevant to the particular time interval, and determining the activity under control of the calendar data; iii) receiving via the data network, or otherwise determining, a communication history of the user representative of the user actively participating in one or more electronic data communication sessions during the particular time interval; iv) receiving via the data network second context data submitted by the user via the data network and associated with the particular time interval, wherein the second context data is representative the activity as interpreted by the user; and v) determining a history of the user representative of the user consuming electronic content information during the particular time interval.

The activity may be inferred from the user's entries in an electronic calendar that pertain to the particular time interval. For example, the user's electronic calendar has entries for business meetings, business trips, attending public events, birthday parties, dentist appointments, vacation schedules, etc. Each respective one of these entries may be interpreted as a respective activity scheduled at the day and in the timeslot as indicated in the calendar. An electronic calendar is a software application running at a network-enabled device of the user, e.g., the user's mobile telephone, smartphone or PC. The user's network-enabled device may have been configured to automatically synchronize the electronic calendar with a copy of the electronic calendar maintained at a server of the service provider.

The activity may be inferred from a geographical location of the user during the particular time interval. The geographical location and the particular time interval may then be correlated with, e.g., a pre-determined schedule of public events in a particular geographic region or and/or with one or more entries in the user's electronic calendar as discussed above. Alternatively, or in addition, the geographical location may be indicative of the premises of a particular institute or organization, e.g., a sports arena, a park, a zoo, a lake, etc., from which may be inferred that the user is attending a sports event or sports training session, is out for a stroll or walking the dog, is visiting the zoo, is onboard a boat, etc., respectively.

The geographical location of the user during the particular time interval may be determined on the basis of explicit user-input via a user-interface of a user's network-enabled data communication device and submitted via the data network from the user's network-enabled data communication device. For example, the user submits to the service provider, during the particular time interval or afterwards, an indication of his/her whereabouts during the particular time interval, e.g., “Amsterdam”, or “Congress on Dark Matter, Helsinki”, or “onboard Eurostar to London”, or “birthday party godmother”, etc. The explicit user-input “onboard Eurostar to London” implies that the user is travelling by train using the high-speed railway service interconnecting London, Paris and Brussels. If the time of the day is also known as well as the Eurostar schedule, the geographic location of the user while travelling onboard the train can be determined, more or less reliably. In addition, one of the activities is then: travelling by train. The explicit user-input “birthday party, godmother”, serves to infer the geographic location as the residence of the user's godmother (e.g., via the user's electronic address book), as well as the activity: attending a party.

The geographical location of the user may also be determined in real-time, e.g., via determining the geographical location of the user's network-enabled mobile communication device connected to the biosensor. For example, in case the network-enabled mobile communication device comprises a mobile telephone, the geographical location of the mobile telephone is determined by the base stations in the mobile telephony network. The mobile telephone network may then upload data, representative of the geographic location, to a pre-determined address of the service provider on the data network, together with an identifier of the mobile telephone, so as to be combined with the biosensor data at the provider of the service. As another example, the geographical location of the mobile communication device is determined by one or more signals received by the mobile communication device from one or more dedicated beacons installed in a geographical area covering a certain public event and forming the current environment of the mobile communication device. The mobile communication device may then upload data, representative of the received signals, together with the biosensor data. As yet another example, the mobile communication device accommodates, or is connected to, an electronic navigational aid such as a GPS receiver. The mobile communication device may then upload data, representative of the GPS data received, together with the biosensor data. A GPS receiver generally does not work indoors as it needs an unobstructed line-of-sight to four or more satellites orbiting the earth. Upon the GPS receiver detecting that the line-of-sight is obstructed, the mobile communication device may attempt to match the last validly obtained geographic location with an entrance of a particular building that can be associated with a particular activity of the user, assuming that the user has intentionally entered the building. The mobile communication device may have an onboard database, generic or tailored to this user's needs, that enables to associate a geographical location with a building. For example, if the building is identified as a fast-food restaurant, it may be assumed that the user will be having a fast meal; if the building is identified as a theater, it may be assumed that the user will be attending a performing-arts event; if the building is identified as a department store, it may be assumed that the user will be busy shopping; if the building has been identified as belonging to a university, and the user is a student, it may be assumed that the user has entered the building in order to do whatever students are supposed to be doing at a university, etc.

The communication history of the user with regard to the user's active participation in one or more data communication sessions may also be used to determine the activity in the particular time interval. Within this context, examples of such a data communication session are: placing or receiving a telephone call or a video call, sending or receiving of an email, sending or receiving of a short-text message (Short Message Service message, or: SMS message; online chat), sending or receiving of a message that includes multimedia content (a Multimedia Messaging Service message, or: MMS message), being involved in real-time texting (RTT), sending or reading text-based posts in a microblogging service such as Twitter, downloading or uploading of an electronic file, receiving or transmitting streaming media, browsing a website or updating one's web page, etc. Consider, for example, the user's mobile telephone. The mobile telephone maintains, or can be configured to maintain, a history of calls placed and calls received, together with the respective telephone numbers of the respective called party or the respective calling party, the time of the beginning and/or the time of termination of the respective call, etc. Consider, as another example, a network-enabled data communication device of the user, e.g., a PC with Internet connection or a smartphone. Such a network-enabled data communication device maintains, or can be configured to maintain, a history of emails sent and emails received, together with the time of the day of sending or receiving, identifiers of the addressees or of the senders, topics of the emails as indicated in their subject-headers, etc. Such a network-enabled device also maintains, or can be configured to maintain, a history of electronic files uploaded or downloaded, streamed, browsed, together with identifiers of the sources or destinations, file type, title of the file, etc. Accordingly, the data communication equipment of the user is, or can be, configured for maintaining a communication history of the user containing information about the active participation of the user in one or more data communication sessions. The qualifier “active” in the feature “active participation” is meant to convey the meaning that the user is knowingly taking part in the data communication sessions so as to be mentally exposed to the data communication session and to the electronic content exchanged. If the user remained unaware of the data communication session going on, it would not be possible to infer a causal relationship between, on the one hand, the emotion or physiological state of the user and, on the other hand, the character of the data communication session. The character of the data communication session may be determined by the semantic aspects of the electronic content exchanged, e.g., the party with whom the user is communicating, one or more topics addressed in the electronic content, the geographic location of the user and the time of the day during the data communication session, etc.

Accordingly, the communication history of the user provides specific details of the activity “communicating” for being included in the activity data so as to be able to specify the activity in more detail. The communication history may be uploaded from the user's data communication equipment to the server of the provider of the service in the invention. Alternatively, the provider of the service in the invention otherwise determines the communication history. For example, the service in the invention forms part of a service package of the user's telecommunication service provider or the user's Internet service provider, as a result of which the provider of the service in the invention has access to at least some of the information about the user's communications as maintained at the user's data communication equipment. As a rule, however, the user's telecommunication service provider and the user's Internet service provider are not allowed to inspect the semantic content being communicated between the user and another party.

The activity of the user may also be determined on the basis of second context data, submitted by the user via the data network and associated with the particular time interval. The second context data is representative of the activity as interpreted by the user. The user may submit the second context data, which pertains to the particular time interval, during the particular time interval, e.g., via the user's smartphone, or afterwards, e.g., from the user's home PC at the end of the day or whenever convenient to the user.

As discussed above with reference to the first context data, the second context data may have been compiled by the user and may take the form of, e.g., a personal text message, a picture or video clip taken by the user's camera or selected from a web page on the Internet, a user-designed icon, etc. The second context data is uploaded during the particular time interval or retroactively. Instead of being compiled by the user, the second context data as submitted by the user is pre-formatted and comprises, e.g., a particular one of a plurality of pre-determined options selectable in a menu accessible through a user-interface, e.g., a graphical user-interface or a speech input user-interface. The user-interface may form part of the network-enabled data communication device that is carried along by the user during operational use of the biosensor. The user-interface may also form part of other network-enabled data processing equipment of the user, e.g., the user's home PC or laptop PC, or the user's smartphone. For example, the user-interface gives access to a menu of a plurality of pre-configured selectable options, and each specific one of the options is representative of a particular activity, such as for example, “work”, “traveling”, “hobby”, “sporting” “relaxing”, “meal”, “study”, “playing the piano”, etc. The representations of the options to the user may be uniform to all users, or may be personalized per specific user. A specific activity associated with a specific one of the options will, however, be considered as uniform to all users, so as to have a frame of reference regarding activities that is common to all users. Accordingly, the second context data is generated by, or under control of, the user and forms an interpretation by the user of the activity of the user.

The activity of the user may also be determined on the basis of a history of the user representative of the user consuming electronic content information during the particular time interval. For example, the user's home entertainment network comprises a PC for browsing the world-wide-web, a play-out device (DVD player, MP3 player, etc.) for playing out pre-recorded electronic content information (e.g., video files such as movies, documentaries, etc., and audio files of music), a TV receiver for receiving TV broadcasts. The web-pages downloaded or the electronic content information streamed via the Internet or played out at a DVD-player or a CD-player or an MP3 player are all instances of electronic content information consumed by the user. Hooked up in the home network, the network can be configured to maintain a history of the electronic content information consumed by the user over time. Accordingly, details of the activity of the user (i.e., details of the electronic content information consumed by this user) can be extracted from the consumption history, such as genre, title, topical aspects of the electronic content information consumed as well as the time intervals wherein the electronic content information was consumed. The consumption history may be uploaded to the service provider. The user may be wearing a mobile biosensor generating time-stamped biosensor data that can be correlated with the time-intervals registered in the consumption history as received at the service provider. Alternatively, or in addition, the home entertainment system has a video camera to capture the user while consuming the electronic content information, and pattern recognition software running on a computer in order to determine an emotional state of the user. Such software is known in the art. For background on such software and computer vision systems see, e.g., “Coding, Analysis, Interpretation, and Recognition of Facial Expressions”, Irfan A. Essa, Georgia Institute of Technology, Graphics, Visualization and Usability Center, Technical Report # GIT-GVU-98-23, August 1998, of which an abridged version appeared in IEEE Transactions on Pattern Analysis and Machine Intelligence, Vol. 19 (7), July, 1997; and “Emotion Recognition Using PHOG and LPQ features”, Abdinav Dhall et al., 2011 IEEE International Conference on Automatic Face & Gesture Recognition (FG 2011), Santa Barbara, USA, March 2011. The home entertainment network may then be configured to identify the user from the images captured by the camera and to determine the emotional state of the user from these images, and to have the emotional states time-stamped and uploaded to the service provider so as to have the various emotional states correlated in the history log with the electronic content consumed.

An embodiment of the method of the invention comprises: receiving via the data network a request for access to the history log; determining whether or not the request is authorized; providing the access via the data network in dependence on the request being authorized.

In this embodiment, the user's history log is used as a personal diary or journal. The user may authorize another person to access the user's history log. The authorization may have been configured to grant to the other person access to the history log in its entirety. Alternatively, the authorization may have been configured to grant to the other person only access to one or more specific portions of the history log, e.g., portions representative of the emotion data and the activity data pertaining to one or more specific time intervals, or portions with activity data representative of one or more pre-determined activities, etc. The authorization may be implemented in a variety of ways.

For example, the user has provided an access code or log-in code to another person for authorizing access to the user's history log via the data network. Different access codes may be given to different persons so as to be able to keep track of who did actually access the history log and when. A different access code per different authorized person also enables to implement access conditions. A first authorized person may be given permanent authorization to access the complete history log. A second authorized person may be given permanent authorization for accessing only particular portions of the history log, e.g., characterized by one or more pre-determined types of activities as logged, or characterized by temporal aspects such as time of the day or day of the week, or characterized by type or valence of the emotional state of the user, etc. A third person is given temporary access to the history log, and only with regard to one or more pre-specified activities as logged.

As another example, the authorization is implemented by means of receiving an invitation from the user, e.g., per email or SMS, with an access code for use only once.

Another embodiment of the method of the invention comprises generating a recommendation with regard to a future activity under control of at least one of: previous emotion data recorded in the history log and representative of one or more previous emotional states of the user and associated with one or more pervious time intervals, and previous activity data recorded in the history log and representative of one or more previous activities of the user associated with the one or more previous time intervals.

In this other embodiment, the history log of the user is used as input to a recommender system run by a recommender service. Recommender systems are known in the art and are configured for recommending items of content information (e.g., books, movies, music, TV programs, web pages, etc) or social or communitarian items (e.g., certain people, certain public events) that are likely to be of interest to the person addressed by the recommender system. A typical recommender system seeks to match a profile of the person with a reference so as to predict the preferences of the person with regard to items not yet considered by the person. The history log of an individual user gives information about the evolution of this user's life style in terms of this user's activities and this user's emotional states in temporal relationship with the activities. The recommender system may recommend activities to this user based on patterns detected in the history log via email, SMS or pre-recorded voice mail, etc. For example, the emotional state of the user has typically changed from a negative valence to a positive valence upon the user's starting to get involved in a certain activity, or the intensity of the positive emotional state typically increased upon the user's starting to get involved in a certain activity. If the service of the invention identifies a trend in the current and most recent emotional states of the user towards a negative emotion, the recommender service may suggest to this user to get involved in the specific activity. Alternatively, if a current pattern of activities resembles past patterns of activities as recorded in the history log and the past patterns resulted in the user eventually assuming a negative emotional state, the recommender system may issue a gentle warning and identify an alternative activity to break away from the current pattern.

In a further embodiment of the method of the invention, the recommendation is generated under combined control of a profile of the user and a further profile of a further person.

In this further embodiment, the history logs of one or more users may serve as a reference to one or more further persons having subscribed to the recommender service. The subscriber may, but need not, be a particular user whose physiological state is being monitored by a biosensor as described earlier. A reference profile is made on the basis of interests, preferences or dislikes declared in advance by one or more users. Alternatively, or in addition, a reference profile is made on the basis of similarities in patterns of activities in temporal correspondence with emotional states as recorded in one or more history logs. A profile of the subscriber is formed so as to be comparable to the reference profile. The subscriber profile is formed, for example, on the basis of interests, preferences or dislikes declared by the subscriber in advance. The recommender service analyzes the history logs, i.e., the patterns of activities and associated emotional states of the users in the reference profile. The recommender service may then issue one or more recommendations to the subscriber in order to recommend one or more activities to the subscriber that, according to the reference profile, is or are likely to cause the subscriber to assume a positive emotional state. The recommendation may be provided to the subscriber electronically (e.g., per email, SMS, pre-recorded voice message, etc.), for example, once a day. Alternatively, or in addition, recommendations are provided to the subscriber when the service identifies one or more new activities that have recently been recorded in the history logs, such as watching a recent movie or reading a recently published novel, or attending a recent performing arts event. The one or more new activities have been recorded as associated with positive emotional states of the users that are considered as the reference with regard to this subscriber.

In a further embodiment of the method of the invention, each respective one of a plurality of further users is being monitored by a respective further biosensor configured for generating respective further biosensor data associated with a respective further particular time interval and representative of a respective further physiological state of the respective further user in the respective further particular time interval. The method comprises: receiving the respective further biosensor data via the data network; associating with the respective further biosensor data a respective further emotional state assumed by the respective further user in the respective further particular time interval; recording in a respective further history log respective further emotion data that is representative of the respective further emotional state and that is associated with the respective further particular time interval; determining respective further activity data representative of a respective further activity of the respective further user taking place in the respective particular time interval; and recording in the respective further history log the respective further activity data associated with the respective further particular time interval. The method also comprises: analyzing the history log and the respective further history log for determining if the activity and the further activity are compatible; if the activity and the further activity are compatible, analyzing the history log and the respective further history log for determining if the emotional state and the respective further emotional state are compatible; and if the emotional state and the respective further emotional state are compatible, notifying the user and the respective further user of their having compatible profiles.

In this embodiment of the method, each specific one of the history logs serves as the profile of the specific user in terms of a history of activities of the specific user in temporal relationship with a history of emotional states of the specific user. The specific profile is then considered as the emotional response of the specific user to a particular activity of this specific user or to a temporal sequence of activities of this specific user. The method of the embodiment attempts to find similarities between the profiles of different users. If different users turn out to have similar emotional responses to compatible activities or to compatible sequences of activities, the method notifies these different users of their emotional compatibility so as to be able to contact each other. The embodiment of the method therefore serves as a tool in order to find soul mates. For example, if the profiles of two users indicate that they develop positive emotional states while watching movies of a particular genre, or movies with a particular cast, it might well be that these users would like to communicate, e.g., via email or microblog, with each other about these movies, or would even like to meet face to face and watch a new movie together.

The invention also relates to network-enabled equipment that comprises: a network-interface for data communication with a data network; a first node for receiving, from a biosensor, biosensor data that is representative of a physiological state of a user of the network-enabled equipment in a particular time interval; and at least one of: a second node for receiving from a navigational aid geographical data representative of a geographical location of the user during the particular time interval; a third node for receiving, from an electronic calendar, calendar data representative of an entry into an electronic calendar of the user and relevant to the particular time interval; a fourth node for receiving a communication history of the user representative of the user actively participating in one or more electronic data communication sessions during the particular time interval and, as an option, conducted via the network-enabled equipment). The network-enabled device further has a user-interface configured for: generating, in response to a first user-interaction with the user-interface, first context data that is representative of an emotional state of the user in the particular time interval as interpreted by the user; and generating, in response to a second user-interaction with the user-interface, second context data that is representative of an activity of the user during the particular time interval as interpreted by the user. The network-enabled device is configured for uploading to a pre-determined address on the data network the biosensor data and at least one of: the geographical data, the calendar data, the communication history, the first context data, and the second context data.

Accordingly, the network-enabled equipment of the invention is configured for gathering data and uploading the data to a server on a data network, in order to for the server to compile a diary or journal of the user and/or to compile a reference profile for use in a recommender service as discussed above. The network-enabled equipment of the invention may be implemented by means of a smartphone or a mobile telephone, configured through, among other things, installing suitable control software and hooking up a biosensor, in order to provide the functionality of the invention specified above. Alternatively, the network-enabled equipment is formed by a conglomerate of data processing devices and apparatus of the user, each specific one thereof for generating or maintaining a specific kind of information such as the geographical data, or the communication history, or the electronic calendar, etc. The conglomerate of data processing devices and apparatus may form a network for exchanging or forwarding the information from the one to the other, and eventually, via the network-interface to the service provider's server on the data network.

The invention also relates to a personal electronic device. The personal electronic device comprises a first node for receiving, from a biosensor, biosensor data that is representative of a physiological state of a user of the network-enabled device in a particular time interval; and at least one of: a second node for receiving from a navigational aid geographical data representative of a geographical location of the user during the particular time interval; a third node for receiving, from an electronic calendar, calendar data representative of an entry into an electronic calendar of the user and relevant to the particular time interval; a fourth node for receiving a communication history of the user representative of the user actively participating in one or more electronic data communication sessions during the particular time interval and conducted via the personal electronic device. The personal electronic device further has a user-interface configured for: generating, in response to a first user-interaction with the user-interface, first context data that is representative of an emotional state of the user in the particular time interval as interpreted by the user; and generating, in response to a second use-interaction with the user-interface, second context data that is representative of an activity of the user during the particular time interval as interpreted by the user. The personal electronic device also comprises data storage. The personal electronic device is configured for maintaining a history log by means of storing in the data storage the biosensor data and the at least one of: the geographical data, the calendar data, the communication history, the first context data, and the second context data.

The personal electronic device combines the data gathering functionality of the network-enabled device, specified earlier above, and the recording functionality, specified earlier above as carried out a server on a data network. The personal electronic device thus keeps the personal data at the personal communication device for privacy reasons, and thus serves as a diary of the user accessible at the personal electronic device. The personal electronic device may, but need not, be a data communication device with a network-interface. For example, a smartphone or mobile telephone can be configured through, among other things, installing suitable control software and hooking up a biosensor, in order to provide the functionality of the personal electronic device specified above.

An embodiment of the personal electronic device is configured for generating a recommendation with regard to a future activity under combined control of: previous emotion data recorded in the history log and representative of one or more previous emotional states of the user and associated with one or more pervious time intervals, and previous activity data recorded in the history log and representative of one or more previous activities of the user and associated with the one or more previous time intervals.

In above embodiment, the personal electronic device is configured, e.g., via installing suitable control software, for analyzing the pattern of activities of the user and the accompanying emotional states of the user. Upon detecting a trend in the emotional states from positive to negative, the personal electronic device may compare the recent activities and recent emotional states to emotional patterns in the past and derive pointers for recommendations to turn the emotional tide.

In an embodiment, the personal electronic device comprises a display monitor and/or a loudspeaker or earphone in order to enable the user to consume electronic content information. Examples of consuming electronic content information have been discussed above: browsing the world-wide-web, playing out a video file or an audio file, receiving video or audio being streamed over the Internet, editing one's microblog, etc. The personal electronic device is configured for logging this user's activity data by way of maintaining a history of this user consuming electronic content information. The personal electronic device is configured to put the biosensor data into temporal relationship with the electronic content information consumed so as to form the history log local to the personal electronic device. Alternatively, or in addition, the personal electronic device may also comprise a specific biosensor in the form of a camera and pattern recognition software for identifying the user and for determining the emotional state of the user from the images captured by the camera.

### BRIEF DESCRIPTION OF THE DRAWING

The invention is explained in further detail, by way of example and with reference to the accompanying drawing, wherein:

FIG. 1 is a block diagram of a system in the invention;

FIG. 2 is a block diagram of a personal electronic device in the invention; and

FIG. 3 is a block diagram of network-enabled data communication equipment of a user.

Throughout the Figures, similar or corresponding features are indicated by same reference numerals.

### DETAILED EMBODIMENTS

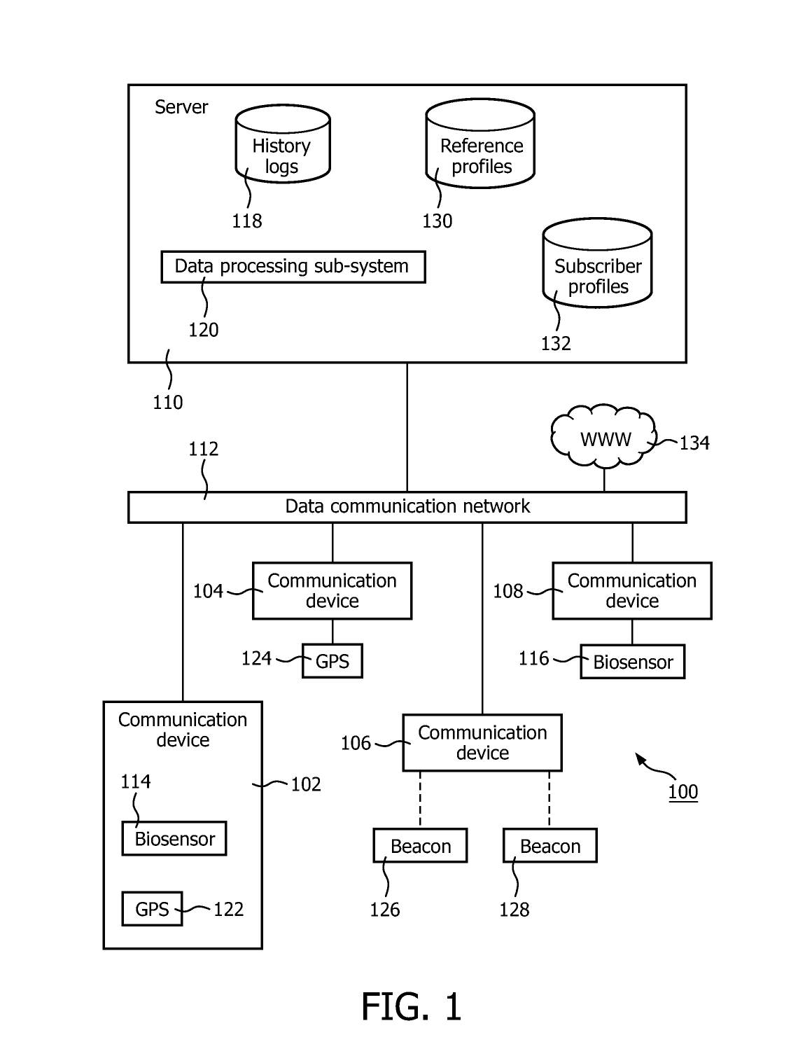

FIG. 1 is a block diagram of a system 100 in the invention. The system 100 comprises a plurality of electronic communication devices, e.g., a first communication device 102, a second communication device 104, a third communication device 106 and a fourth communication device 108. In the system 100 of FIG. 1, each of the plurality of communication devices implements the functionality of the network-enabled equipment as specified in the appended claims. The system 100 also comprises a server 110 and a data network 112, e.g., the Internet. Each respective one of the plurality of communication devices has a network-interface (not shown) configured for data communication between the respective communication device and the server 110 via the data network 112.

Each respective one of the plurality of communication devices accommodates a respective biosensor sub-system, or is configured for receiving biosensor data from a respective external biosensor sub-system. For example, the first communication device 102 accommodates a first biosensor sub-system 114, whereas the fourth communication device 108 is connected to a fourth biosensor sub-system 116 external to the fourth communication device 108. The second communication device 104 accommodates, or is connected to, a second biosensor sub-system (not shown) and the third communication device 106 accommodates, or is connected to, a third biosensor sub-system (not shown).

Each respective one of the biosensor sub-systems, mentioned above, comprises one or more biosensors. As known, a biosensor is a type of sensor that is used in the fields of biotelemetry and of remote physiological monitoring. A biosensor is configured for sensing one or more physiological parameters to produce an output signal representative of the values of the one or more physiological parameters sensed. Examples of a physiological parameter are: a heart rate or pulse rate of the user, blood pressure, galvanic skin response, body temperature, skin temperature, heat flow, respiration rate, etc. Accordingly, the output signal of a biosensor is representative of a physiological state of the person being monitored by the biosensor.

Each respective one of the plurality of electronic communication devices is configured for uploading to the server 110 biosensor data that is representative of a physiological state, as sensed, of a respective user of the respective electronic communication. Preferably, the biosensor data is time-stamped or otherwise put into a temporal context so as be able to associate the biosensor data with the physiological states of the user assumed during certain time periods. The server 110 stores the respective biosensor data, received from a respective one of the plurality of electronic communication devices, in a respective history log at data storage 118.

The biosensor data is uploaded, e.g., in real-time, i.e., upon the biosensor data having being generated from the output signal from the relevant biosensor sub-system. For example, the biosensor data is generated continuously and is uploaded to the server 110 continuously. As another example, the biosensor data is generated continuously only during a specific time-interval or during a series of disjoint time-intervals, and is uploaded to the server 110 continuously during such a time interval. The disjoint time-intervals may have been scheduled in advance, or each such time-interval may be started upon request from the server 110. As yet another example, the biosensor data is generated either continuously or intermittently, and is only uploaded to the server 110 in real-time upon the relevant biosensor sub-system detecting a significant change in the physiological state of the user of the relevant communication device. That is, the biosensor data uploaded is representative of the changes in the physiological state.

Alternatively, the biosensor data is uploaded to the server 110 in a delayed manner. For example, the biosensor data generated during a sequence of time-intervals is accumulated and stored locally at the relevant biosensor sub-system, or stored locally at the relevant communication device, on an ongoing basis, e.g., continuously, intermittently, or upon detection of a significant change in the physiological state of the user. The most recently accumulated biosensor data may then be uploaded to the server 110, e.g., once every day, once every week, upon a user-command or upon request from the server 110, etc.

Note that it is not necessary to have the relevant biosensor sub-system accommodated at, or permanently connected to, the relevant communication device. The biosensor sub-system of a user may then form a dedicated entity that is configured for offloading the locally stored biosensor data to another device, e.g., the communication device of the user or a data storage device such as a USB-stick. The biosensor data thus received by the other device may then be transferred to the server 110 later on via the communication device of the user and the data network 112.

Each individual one of the plurality of electronic communication devices can be a mobile communication device or a stationary communication device. Examples of a mobile communication device are a mobile telephone or a smartphone, a personal digital assistant (PDA) or palmtop PC with a network-interface, a laptop PC with a network-interface. Each of these examples is representative of a general-purpose type of communication device that is configured for wireless data communication via the data network 112. Another example of a mobile communication device is a dedicated device that is configured for wireless communication of biosensor data generated by an onboard biosensor sub-system. Examples of a stationary communication device include a desktop PC with a network-interface, a laptop PC with a network-interface, a home-network with a network-interface.

The server 110 comprises a data processing sub-system 120. The data processing sub-system 120 is configured for processing the biosensor data of a specific user for determining an emotional state assumed by the user in a particular time interval or the emotional states assumed by the user in two or more particular time intervals.

As mentioned earlier, the emotional state of a person may be determined from the biosensor data that are representative of the values of one or more physiological parameters sensed during a particular time interval.

However, the values of the physiological parameters typically indicate an intensity of an emotion, but not necessarily the valence of the emotion. Optionally, therefore, first context data may be used to ascertain the emotional state of the user associated with the biosensor data of the user as pertaining to a particular time interval.

The first context data may be formed by explicit user-input entered via, e.g., a user-interface (not shown) of his/her communication device or a user-interface (not shown) of his/her biosensor sub-system. The explicit user-input is configured so as to be indicative of this user's current emotion. For example, the user-interface may provide a menu of a plurality of pre-configured selectable options, each particular one of the options being indicative of a particular one of a plurality of emotions. The options may be presented in a graphical part of the user-interface, for example, as icons or graphical symbols, as colors or as words or expressions in a written natural language. Selecting a particular one of the options from the menu generates the first context data representative of the particular emotion associated with the particular option as selected by the user. As another example, the user-interface is configured for speech-to-data conversion, and the user may enter a voice command indicative of his/her currently emotion as felt.

The first context data is associated with a particular time interval through a temporal indication included in the context data, e.g., through time-stamping or through explicit user-input specifying the particular time-interval. Upon receipt of the respective first context data from a respective one of the plurality of communication devices, the server 110 stores the first context data in the respective one of the history logs at data storage 118. The temporal indication of the respective first context data enables to associate the respective first context data with the biosensor data received from the respective communication device and pertaining to the same time interval as the first context data. Accordingly, a respective history log at data storage 118 is a representation of emotional states assumed over time by the respective user of the respective communication device.

The duration of a specific emotional state of an individual user, i.e., the length of time over which the individual user can be considered to be in this specific emotional state, depends on, among other things, the personality of this individual user and this individual user's perception of the changes in the world around him/her. The duration also depends on the definition of the concept “emotional state” and on the number of different emotional states considered to cover the entire emotional spectrum believed to be applicable to an individual. In addition, it is not always clear where one emotional state ends and where another one begins, or whether or not an individual can only assume a single emotional state at a single moment.

In the invention, a particular emotional state, determined to be applicable to an individual in a particular time interval, is sought to be brought into relation with a particular activity of this individual in this particular time interval, or with the particular activity in this particular time interval and one or more further activities in one or more further time intervals preceding the particular time interval. As will be discussed later, a recorded history of the user's emotions and activities can be used as a diary or as a microblog with emotional annotation, or the recorded history can be used as a basis for a recommender system implemented by the data processing system 120. As known, a recommender system is a system for processing and filtering information in order to generate recommendations to one or more individuals with regard to information items (specific books, specific movies, specific music, specific web pages, etc.), social items (specific events, specific social groups, specific persons), health-related issues, dietary issues, etc., etc., that are likely to be of interest or benefit to the one or more individuals. Accordingly, data pertaining to the user's activity need to be provided to the server 110 so as to be able to tie an emotional state of a user to one or more activities of the user.

The activity of a specific user of a specific one of the communication devices during a particular time interval can be determined in a variety of manners.

For example, a user's activity may be inferred by the data processing sub-system 120 from his/her geographical location or from his/her geographical location and the rate of change of the geographical location. In a first scenario, the first communication device 102 has an onboard first navigational aid 122 that keeps track of the first user's geographical location, and the second communication device 104 is connected to a second navigational aid 124 external to the second communication device 104. The geographical location of, e.g., the first user in relation to time can be used to infer activities of the first user. If the first user stays around a specific geographical location coinciding with a restaurant for some time, it may be plausible to conclude that the first user is having a meal or a coffee. If the first user stays within a specific geographical area coinciding with a specific venue (e.g., sports stadium, theme park, museum, hiking grounds, shopping mall, office building, flea market) for some time, it may be concluded that the first user is involved in an activity associated with the specific venue. If the geographic location of the first user is changing with time so as to trace out a route on a map that coincides with, e.g., an expressway, a railroad track, a bicycle path, a hiking path, etc., it may be concluded that the first user is travelling. As discussed above with respect to the biosensor data, geographical data that is representative of the geographical location(s) of the first user may be uploaded on an ongoing basis, or only intermittently, or upon a change in geographical location that merits an update of the current geographical location at the server 110. Alternatively, a most recent history of time-stamped geographical locations is stored locally at the first navigational aid 122, or elsewhere at the first communication device 102, and is uploaded periodically, or upon an explicit user-input to the first communication device 102, or upon request from the server 110, etc.

A second scenario, similar to above first scenario, is applicable to the second user of the second communication device 104 that does not have an onboard navigational aid, but is instead connected to the second navigational aid 124 external to the second communication device 104.

In a third scenario, the third communication device 106 is configured to interface with a plurality of radio beacons: a first radio beacon 126, a second radio beacon 128, etc., distributed across a geographical area, e.g., a theme park, a shopping mall, an art show, a district of a town, etc. A geographical location of the third communication device 106 can then be determined based on the relative position of the third communication device 106 with respect to one or more of the radio beacons. For example, the third communication device 106 is configured to register the presence of a particular one of the radio beacons if the particular radio beacon is within proximity of the third communication device 106. Alternatively, the third communication device 106 is configured to determine a relative position of the third communication device 106 with respect to the radio beacons based on signal strength of the radio signals received. As discussed above with respect to the biosensor data, data that is representative of the geographical location(s) of the third user may be uploaded on an ongoing basis, or only intermittently, or upon a change in geographical location that merits an update of the current geographical location at the server 110. Alternatively, a most recent history of geographical locations is stored locally at the third communication device 106, and is uploaded periodically, or upon an explicit user-input to the third communication device 106, or upon request from the server 110, etc.

The activity of a user of a specific one of the communication devices during a particular time interval can also be determined from, e.g., a calendar of this user. For example, the fourth communication device 108 comprises an electronic calendar (not shown) of the fourth user to mark a particular date in the future with one or more particular entries such as appointments, events, actions, reminders, etc., scheduled at or pertaining to that particular date. Data representative of the calendar markings may then be uploaded from the third communication device 106 to the server 110. The data processing sub-system 120 correlates the data representative of the calendar markings with the biosensor data from the fourth biosensor sub-system 116 to establish temporal relationships between emotional states and activities.

The activity of a user of a specific one of the communication devices during a particular time interval can also be determined from, e.g., explicit user-input to the specific communication device to generate second context data. For example, the specific communication device enables the user to select via the communication device's user-interface (not shown), a specific option from a menu of options, the specific option being representative of a specific activity. Selection of the specific option prepares the second context data, representative of the specific activity to be uploaded to the server 110 or to be stored locally at the specific communication device together with a time stamp, for being uploaded to the server 110 later on. The data processing sub-system 120 then processes the received second context data representative of the activity and adds this data to the relevant history log as associated with the relevant time interval.

Accordingly, the data storage 118 accumulates biosensor data and/or data on the emotional states of each individual user, as well as further data about activities of each individual user and stores this data in an individual history log. The history logs can now be used in a variety of application scenarios of providing a service on the data network 112. Examples of application scenarios will be discussed below and will be illustrated, by way of example, with reference to the first user, i.e., the user of the first communication device 102. It will be clear that the application scenarios may be applicable to any of the other users of the other communication devices.

In a first application scenario, the server 110 maintains a history log for the first user in the data storage 118. The history log of the first user represents a history of this first user with regard to emotional states of the first user and activities of the first user put into a temporal relation with each other. For example, the first user's activities are represented in the history log as providing a context for the emotional states of the first user. The first user may upload, via the first communication device 102 or another authorized network-enabled device of this first user, additional annotation data that provides additional information about the first user's emotional state and/or the first user's activities. The additional annotation data is added to the first user's history log so as to enhance or embellish the context of the emotional states. For example, the first user uploads to the server 110 one or more text entries, pictures, video clips or audio clips that are associated with a particular activity of the first user. The additional data uploaded may get time-stamped by the server 110 and written to the relevant history log as associated with the current activity and current emotional state of the first user. Alternatively, the first user may specify to the server 110 that the additional data uploaded is to be associated retroactively with a specific time interval in the first user's history log or with a specific activity of the first user in the past. In this manner, the first user's history log serves as a personal diary or a personal journal to the first user. The personal diary or personal journal at the server 110 can be consulted by the first user via the data network 112, and may be edited afterwards via the data network 112.

The first user may authorize one or more other persons to access his/her personal diary as maintained in the history log of the first user at the data storage 118. For example, the first user authorizes his/her spouse to access the entire content of his/her personal diary at any time. The first user authorizes a friend to access only one or more specific portions of his/her personal diary at any time or only for a pre-determined length of time. A specific portion of the diary may be characterized by one or more attributes that serve to identify the specific portion in the history log. such as, e.g., a temporal aspect, a geographical aspect, an emotional aspect or a semantic aspect of the entries in the history log. For example, the specific portion relates to a certain specific day in the past or a certain specific hour on a specific day in the past. As another example, the specific portion relates to a geographical location, e.g., a site visited by the first user and recorded as an activity in the history log. As another example, the specific portion relates to one or more specific activities of the first user as recorded in the history log, e.g., the first user's weekly tennis games. As yet another example, the specific portion relates to a specific emotional state of the first user. As still another example, the specific portion relates to one or more specific characteristics of the uploaded additional data, e.g., a specific keyword occurring in one or more text entries, or a specific keyword occurring in one or more identifiers of the pictures, audio clips, video clips, etc.

The first user may authorize the other person by transmitting via the data network 112 an invitation to a network-enabled device of the other person. The invitation is, preferably, pre-formatted and contains authorization data configured for authorizing the other person to access the first user's history log as maintained at the server 110. Preferably, the invitation is an electronic invitation with an embedded script to automate, or at least facilitate, the other person's procedure for accessing the first user's history log at the server 110. The other person contacts the server 110 via the data network 112 by submitting the authorization data. The data processing sub-system 120 of the server 110 verifies the validity of the authorization data and verifies the identity of the other person contacting the server 110. Optionally, the data processing sub-system 120 may request the first user to confirm having given the authorization to this other person. The data processing sub-system 120 grants access to the other person for accessing the first user's history log, or one or more specific portions thereof, if the authorization is found to be valid, and denies the access if the authorization is found to be invalid. Access control technologies, including identification processes, authentication processes and authorization processes, are well known in the field of computer security and will not be discussed here in further detail.

In a second application scenario, the server 110 maintains a first database 130 of reference-profiles, and a second database 132 of subscriber-profiles.

Each specific one of the reference-profiles in the first database 130 is representative of a specific set of human characteristics of one or more of the users of the communication devices, among which are the first user of the first communication device 102, the second user of the second communication device 104, the third user of the third communication device 106, the fourth user of the fourth communication device 108, etc.

The reference-profiles in the first database 130 may have been pre-determined. For example, the users of the communication devices have been asked to explicitly declare their interests, preferences and dislikes, with regard to their life style. The reference-profiles in the first database 130 are then formed on the basis of the declared interests, preferences and dislikes of the users of the communication devices. Alternatively, the reference-profiles in the first database 130 are formed on the basis of the history logs in the data storage 118. For example, a particular reference-profile in the first database 130 is formed by one or more common denominators of characteristics of each individual one of a particular sub-set of the users of the communication devices, e.g., one or more particular activities as recorded in their history logs, or particular patterns in their emotions as correlated to their activities, etc.

Each specific subscriber-profile in the second database 132 is a profile of a subscriber to a recommendation service provided by the server 110. A particular subscriber-profile in the second database 132 may, but need not, be a subscriber-profile of a particular user of a particular one of the communication devices. When subscribers register with the recommendation service of the server 110, they are requested to declare their interests, preferences and dislikes, with regard to their life style.

In the recommendation service at the server 110, the data processing sub-system 120 seeks to match a particular sub-scriber profile with one or more reference-profiles as stored in the first database 130. Each specific one of the reference-profiles in the first database 130 stands for one or more history logs wherein activities have been correlated with emotions. A matching reference-profile is associated with one or more history logs in data storage 118. The one or more history logs of the matching reference-profile comprise information about specific activities that gave rise to positive emotional states. The server 110 may then suggest to the particular subscriber such specific activities or similar activities. For example, the server 110 searches the world-wide-web 134 for events (e.g., sports events, performing arts events, exhibitions, festivals, flea markets) which have been scheduled to occur in the near future within a geographical range of the particular subscriber and whose nature corresponds to one or more of the specific activities that gave rise to a positive emotional state according to the matching reference-profile. The server 110 then sends notifications of such events to the particular subscriber via the data network 112. As another example, the server 110 searches the one or more history logs associated with the reference-profile for the occurrence of pointers to specific content information (e.g., books, movies, music, video games, etc.), and/or specific hardware items (e.g., toys, collectibles, tools, etc.), and/or to specific actions (e.g., hiking in a specific area, dining at a particular restaurant, participating in a particular sports game, taking up a certain hobby) related to the recorded activities that are associated with a positive emotional state according to the reference-profile.

If the particular subscriber is actually a particular one of the users of the communication devices, the data processing sub-system 120 may search for patterns in the evolution of the emotional state of this particular subscriber occurring in dependence on activities of this particular subscriber as recorded in this subscriber's history log in data storage 118. Assume that the data processing sub-system 120 identifies a recent occurrence of a downward trend in the intensity of a positive emotional state of the particular subscriber, a recent occurrence of an upward trend in the intensity of a negative emotional state, or that the data processing sub-system 120 determines a recent switching from a positive emotional state of the particular subscriber to a negative emotional state of the particular subscriber. The data processing sub-system 120 may then analyze the one or more history logs associated with the reference-profile for clues that may help this particular subscriber to regain a more positive emotional state. For example, it may turn out that one or more history logs of the reference-profile contain patterns of emotional trends similar to the one of the particular subscriber, against a background of activities similar to those of the particular subscriber. The data processing sub-system 120 may then analyze the subsequent course of affairs in each of the history logs of the reference-profile in order to find pointers to specific content information, hardware items or actions that may help the particular subscriber to reach a more positive emotional state. If such pointers are found, the server 110 sends a recommendation to the particular subscriber, e.g., via the data network 112, indicating the specific content information, hardware items or actions. The data processing sub-system 120 may thereupon monitor the emotional state of this particular subscriber in order to check whether or not the recommendations have had a positive influence on the emotional state. If so, this may then reinforce the relationship between the patterns of emotional trends and the helpfulness of the pointers found with regard to this particular subscriber. The pointers found may then be added to the profile of this particular subscriber as maintained in the second database 132 for future reference by the data processing sub-system 120.

Alternatively, or in addition, the data processing sub-system 120 may analyze the history log of this particular subscriber for clues that may help the particular subscriber to regain a more positive emotional state. For example, it may turn out that the history log of this particular subscriber contains one or more patterns of emotional trends that occurred in the past and that are similar to a current pattern of emotional trends against a background of current activities comparable to those associated with the past trends. The data processing sub-system 120 may then analyze the subsequent course of affairs in the history log of the particular subscriber in order to find pointers to specific content information, hardware items or actions that may currently help the particular subscriber to reach a more positive emotional state. If such pointers are found, the server 110 sends a recommendation to the particular subscriber, e.g., via the data network 112, indicating the specific content information, hardware items or actions. The data processing sub-system 120 may thereupon monitor the emotional state of this particular subscriber in order to check whether or not the recommendations have had a positive influence on the emotional state. If so, this may then reinforce the relationship between the patterns of emotional trends and the helpfulness of the pointers found with regard to this particular subscriber. The pointers found may then be added to the profile of this particular subscriber as maintained in the second database 132 for future reference by the data processing sub-system 120.

The system 100 in the diagram of FIG. 1 forms a client-server architecture for providing a diary service to a population of users of communication devices, or a recommendation service to a population of such users and/or to subscribers to such service who are not participating in the uploading of biosensor data.

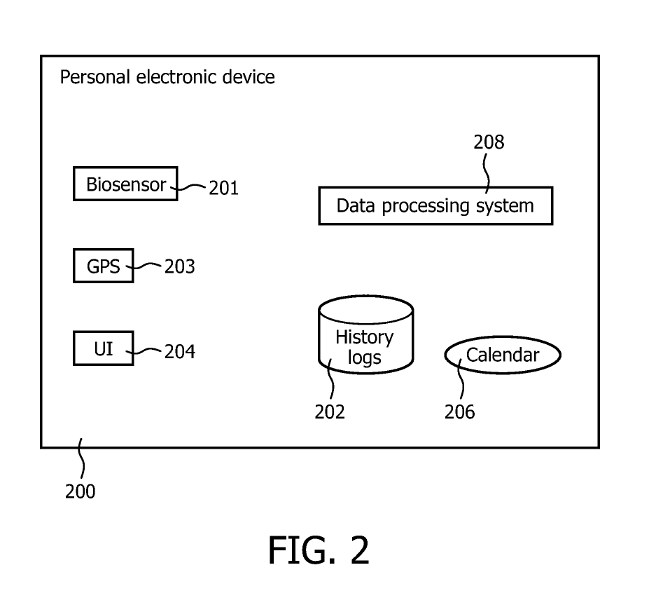

FIG. 2 shows a block diagram of a personal electronic device 200 that performs the data gathering functionality of an individual one of the communication devices discussed above with reference to the system 100 of FIG. 1, as well as the keeping of a personal journal and/or providing of a recommendation service to the user of the personal electronic device 200. Accordingly, the biosensor data, the emotion data and the activity data need not leave the personal electronic device 200.

The personal electronic device 200 comprises a biosensor 201 (or an input to connect to a biosensor) for producing the biosensor data, as discussed above. The personal electronic device 200 is configured to store the biosensor data in a history log 202 at the personal electronic device 200, together with an indication pertaining to the particular time interval during which the biosensor data was generated or received and, optionally.

The personal electronic device 200 also comprises components for determining the activity data representative of an activity of the user as pertaining to the particular time interval. For example, the personal electronic device 200 comprises a navigational aid 203 to generate geographical data representative of the geographical location of the personal electronic device 200 and, therefore, of its user, during the particular time interval. The personal electronic device 200 is configured to store the geographical data in the history log 202 at the personal electronic device 200, together with an indication pertaining to the particular time interval during which the geographical data was generated or received. The user may then later on inspect his/her history log 202 and add, via a user-interface 204 of the personal electronic device 200, annotations about an activity. Alternatively, or in addition, the personal electronic device 200 has a software application 206 that implements an electronic calendar at the personal electronic device 200. Entries made as associated with a particular tine interval may then be indicative of one or more activities of the user during this time interval. Alternatively, or in addition, the user specifies to the personal electronic device 200 his/her activities via explicit user-interaction with the user-interface 204. For example, the user-interface 204 provides a menu of pre-configured selectable options, each individual one of which is representative of an individual activity. In response to the explicit user-interaction of the user with the user-interface 204, the personal electronic device 200 generates second context data representative of the activity specified by the user. Accordingly, the personal electronic device 200 acquires activity data about a particular activity of the user pertaining to the particular time interval.

The user may also specify first context data about his/her emotional state to the personal electronic device 200 via explicit user-interaction with the user-interface 204. For example, the user-interface 204 provides another menu of pre-configured selectable options, each individual one of which is representative of an individual emotional state. In response to the explicit user-interaction of the user with the user-interface 204, the personal electronic device 200 generates the first context data representative of the emotional state of the user as interpreted by the user. The biosensor data and the first context data together may then be used to determine the emotional state of the user, as represented by the emotion data. The activity data and the emotion data are logged in the history log 202 as pertaining to a particular time interval. Accordingly, the history log 202 stores data representative of a history of the user of the personal electronic device 200 with regard to emotional states of the user and activities of the user, put into a temporal relation with each other.

The personal electronic device 200 also has a data processing system 208. While the biosensor 201 is monitoring the physiological state of the user, and while the history log 202 is accumulating the emotion data and activity data, the data processing system 208 analyzes the trends in the recent emotional state of the user. If the trend is representative of an emotional state evolving in the direction of a negative valence, or if the intensity of the negative emotion is increasing, the data processing system 208 consults the history log 202 to find matching patterns in emotional trends to identify turning points in the form of certain activities that appeared to have turned the emotional state to one of a positive valence or to a positive emotion with a higher intensity. Alternatively, the data processing system 208 may spot activities that are always associated with a positive emotion in the history log of this user. The data processing system 208 then provides a recommendation to the user of the personal electronic device 200 to undertake one or more of such certain activities that may fit the current set of circumstances of the user, e.g., as may be derived from, e.g., the time of the day, the current geographic location or the entries in the electronic calendar 206.

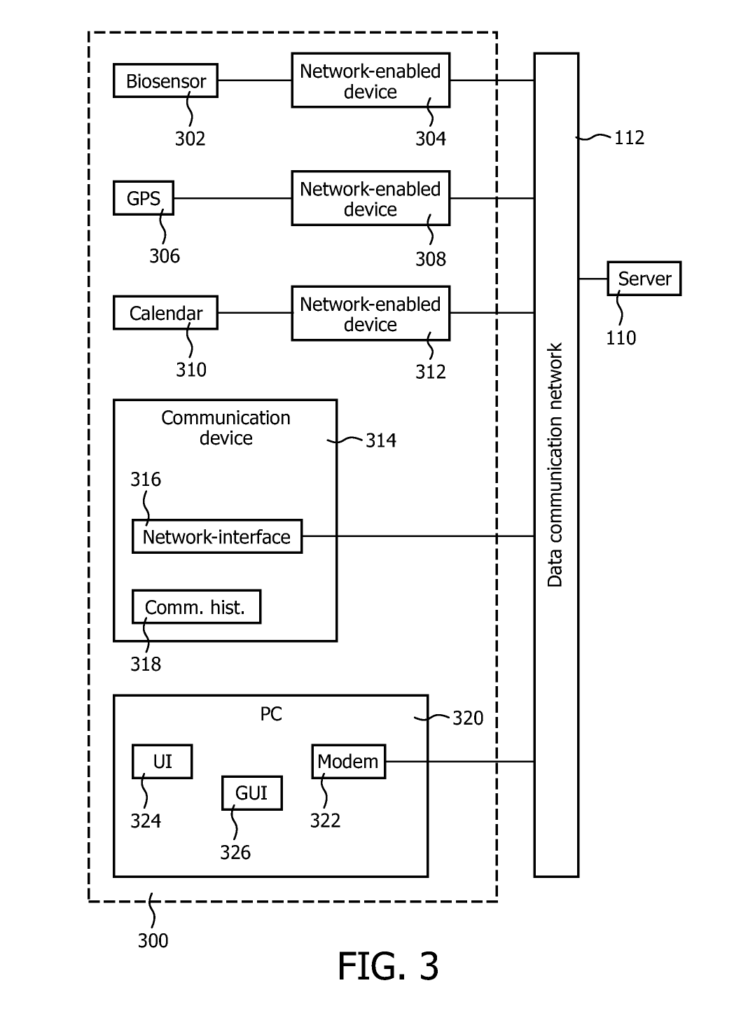

FIG. 3 is a block diagram of network-enabled data processing equipment 300 of a user, wherein the functionalities of the first communication device 102, discussed above with reference to FIG. 1, are distributed between different data processing devices of the user.

In the example shown in FIG. 3, the functionality of the first biosensor sub-system 114 of the first communication device 102 is implemented in the network-enabled data processing equipment 300 by means of a physically separate entity that comprises a biosensor sub-system 302. The biosensor sub-system 302 may be connected to the data communication network 112 via a network-enabled device 304, either permanently, or when the user so desires, in order to upload time-stamped biosensor data to the server 110, either at the time of being generated or in batches of biosensor data accumulated at the biosensor sub-system 302 over time.

The network-enabled data processing equipment 300 of the example illustrated also comprises a separate entity with a functionality to determine the geographical location of the user over a certain time span, e.g., a GPS-receiver 306. The GPS receiver 306 may be connected to the data communication network 112 via a further network-enabled device 308, either permanently, or when the user so desires, in order to upload time-stamped geographical data to the server 110, either at the time of being generated or in batches of time-stamped geographical data accumulated at the GPS receiver 306 over time. As mentioned above, the activities of the user can be inferred, under circumstances, from his/her geographical location(s) over time.

The network-enabled data processing equipment 300 of the example illustrated in FIG. 3 also comprises a separate entity with a functionality of an electronic calendar. The electronic calendar 310 may be connected to the data communication network 112 via another network-enabled device 312, either permanently, or when the user so desires, in order to upload the calendar entries to the server 110. As mentioned above, the activities of the user can be inferred, under circumstances, from the entries made in his/her electronic calendar.

The network-enabled data processing equipment 300 of the example illustrated in FIG. 3 also comprises a separate entity with a functionality of an electronic data communication device 314, e.g., a smartphone. The smartphone 314 has a data-network interface 316 and is configured to maintain a communication history in local storage 316. The communication history comprises information about emails sent by the user or received and opened by the user, telephone calls placed or received by the user, SMS messages sent or received by the user, websites visited by the user while browsing the world-wide-web, etc. etc. As mentioned above, details about activities of the user can be inferred, under circumstances, from the communication history.

The network-enabled data processing equipment 300 of the example illustrated in FIG. 3 also comprises a separate PC 320 that is equipped with a modem 322. The PC 320 has a user interface 324 suitable for text-input (e.g., a keyboard) and a graphical user-interface (GUI) 326 interacted with via, e.g., a computer mouse (not shown). The PC 320 lets the user access his/her history log maintained at the server 110 and lets the user edit or embellish his/her history log with alphanumeric text and with pictures, video clips, audio clips, etc. The PC 320 also lets the user compile and upload the first context data (representative of the emotional state of the user as interpreted by the user) and/or the second context data (representative of the activity of the user, as interpreted by the user).

1. A method of providing a service on a data network, wherein:
a user is being monitored by a biosensor configured for generating biosensor data associated with a particular time interval and representative of a physiological state of the user in the particular time interval;
the method comprises:
receiving the biosensor data via the data network;
associating with the biosensor data an emotional state assumed by the user in the particular time interval;
recording in a history log emotion data that is representative of the emotional state and that is associated with the particular time interval;
determining activity data representative of an activity of the user taking place in the particular time interval;
recording in the history log the activity data associated with the particular time interval;
generating a recommendation with regard to a future activity under combined control of:
previous emotion data recorded in the history log and representative of one or more previous emotional states of the user and associated with one or more pervious time intervals, and
previous activity data recorded in the history log and representative of one or more previous activities of the user and associated with the one or more previous time intervals,
wherein the recommendation is further generated under combined control of a profile of the user and a further profile of a further person and wherein the history log of the further person serves as a reference.

2. The method of claim 1, wherein the associating of the emotional state with the biosensor data comprises:
receiving first context data submitted by the user via the data network and associated with the particular time interval, wherein the context data is representative of the emotional state as interpreted by the user; and
including the first context data into the emotion data.

3. The method of claim 1, wherein the determining of the activity data comprises at least one of:
i) receiving via the data network geographical data representative of a geographical location of the user during the particular time interval and determining the activity under control of the geographical location and the particular time interval;
ii) receiving via the data network calendar data representative of an entry into an electronic calendar of the user and relevant to the particular time interval, and determining the activity under control of the calendar data;
iii) determining a communication history of the user representative of the user actively participating in one or more electronic data communication actions/sessions during the particular time interval;
iv) receiving via the data network second context data submitted by the user via the data network and associated with the particular time interval, wherein the second context data is representative of the activity as interpreted by the user; and
v) determining a history of the user representative of the user consuming electronic content information during the particular time interval.

4. The method of claim 1, wherein the method comprises:
receiving via the data network a request for access to the history log;
determining whether or not the request is authorized;
providing the access via the data network in dependence on the request being authorized.

5-6. (canceled)

7. The method of claim 1, wherein:
each respective one of a plurality of further users is being monitored by a respective further biosensor configured for generating respective further biosensor data associated with a respective further particular time interval and representative of a respective further physiological state of the respective further user in the respective further particular time interval;
the method comprises:
receiving the respective further biosensor data via the data network;
associating with the respective further biosensor data a respective further emotional state assumed by the respective further user in the respective further particular time interval;
recording in a respective further history log respective further emotion data that is representative of the respective further emotional state and that is associated with the respective further particular time interval;
determining respective further activity data representative of a respective further activity of the respective further user taking place in the respective particular time interval;
recording in the respective further history log the respective further activity data associated with the respective further particular time interval;
analyzing the history log and the respective further history log for determining if the activity and the further activity are compatible;
if activity and the further activity are compatible, analyzing the history log and the respective further history log for determining if the emotional state and the respective further emotional state are compatible; and
if the emotional state and the respective further emotional state are compatible, notifying the user and the respective further user of their having compatible profiles.

8. (canceled)

9. A personal electronic device wherein:
the personal electronic device comprises:
a first node for receiving, from a biosensor, biosensor data that is representative of a physiological state of a user of the network-enabled device in a particular time interval;
at least one of:
a second node for receiving from a navigational aid geographical data representative of a geographical location of the user during the particular time interval;
a third node for receiving, from an electronic calendar, calendar data representative of an entry into an electronic calendar of the user and relevant to the particular time interval;
a fourth node for receiving a communication history of the user representative of the user actively participating in one or more electronic data communication sessions during the particular time interval (and conducted via the personal electronic device;
a user-interface configured for:
generating, in response to a first user-interaction with the user-interface, first context data that is representative of an emotional state of the user in the particular time interval as interpreted by the user; and
generating, in response to a second use-interaction with the user-interface, second context data that is representative of an activity of the user during the particular time interval as interpreted by the user; and
data storage;
the personal electronic device is configured for maintaining a history log by means of storing in the data storage the biosensor data and the at least one of: the geographical data, the calendar data, the communication history, the first context data, and the second context data;
the personal electronic device further being configured for generating a recommendation with regard to a future activity under combined control of:
previous emotion data recorded in the history log and representative of one or more previous emotional states of the user and associated with one or more pervious time intervals, and
previous activity data recorded in the history log and representative of one or more previous activities of the user and associated with the one or more previous time intervals,
wherein the recommendation is further generated under combined control of a profile of the user and a further profile of a further person and wherein the history log of the further person serves as a reference.

10. (canceled)
